(19) United States
(12) Patent Application Publication
(10) Pub. No.: US 2023/0288395 A1
(43) Pub. Date: Sep. 14, 2023

(54) OXIDATIVE STABILITY TEST METHODS FOR CHEMICALLY RECYCLED PLASTIC FEEDSTOCKS

(71) Applicant: ECOLAB USA INC., St. Paul, MN (US)

(72) Inventors: Ashish DHAWAN, Aurora, IL (US); Carter M. SILVERNAIL, Lakeville, MN (US); Emily Rebecca WOOD, Naperville, IL (US); Kameswara VYAKARANAM, Sugar Land, TX (US); Daniel Robert DREYER, Rosenberg, TX (US); Karina EURESTE, Houston, TX (US)

(73) Assignee: ECOLAB USA INC., St. Paul, MN (US)

(21) Appl. No.: 18/175,919

(22) Filed: Feb. 28, 2023

Related U.S. Application Data
(60) Provisional application No. 63/318,511, filed on Mar. 10, 2022.

(51) Int. Cl. G01N 33/28; G01N 27/06

(52) U.S. Cl. CPC G01N 33/2805; G01N 33/2888; G01N 33/2847; G01N 27/06

(57) ABSTRACT

The present disclosure relates to various testing methods that can be applied to oils, such as pyrolysis oils. Different testing methods can be used to determine the oxidative stability of an oil. The testing methods may also be used to differentiate between various oils and to determine the effectiveness of various antioxidants on the oxidative stability of an oil.

### TECHNICAL FIELD

The present disclosure generally relates to analytical methods used to test oils.

### BACKGROUND

Plastic is the fastest growing waste product and poses a significant environmental problem. Converting waste plastic into useful, higher value products, such as crude oil or feedstock, for the production of olefins in a steam cracker provides an opportunity to deal with the plastic waste problem.

Plastic is primarily made up of polyethylene and polypropylene. Through various processes, such as pyrolysis, the carbon-carbon bonds and carbon-hydrogen bonds of the plastics are broken. The breakdown of the plastic can result in varying types and amounts of the oligomeric chains or monomers high in ethylene, propylene, butadiene, styrene and other unsaturates (e.g., α-ω di-olefins which could have multiple reactive units).

The unsaturated components are inherently unstable and subject to deterioration due to oxidation or the monomers can repolymerize, which can result in gums or sediment within the plastic-derived synthetic feedstocks.

Oxidation and gums can cause problems during the recovery, transport, storage, or use of the synthetic feedstocks. They also cause fouling of process equipment leading to problems, such as plugging and corrosion of the various production units. The precipitated gum-like materials can block filters, pumps, pipelines, and other installations or be deposited in tanks, thus entailing additional cleaning and costs.

### BRIEF SUMMARY

The present disclosure provides various methods for testing oils. In some embodiments, the present disclosure provides a method of determining the oxidative stability of a pyrolysis oil. The method comprises heating a sample of the pyrolysis oil to a temperature from about 50° C. to about 220° C., passing a stream of air through the sample of pyrolysis oil, forming a volatile reaction product, transporting the volatile reaction product into a measurement solution comprising deionized water, and measuring an electrical conductivity of the measurement solution.

In some embodiments, the pyrolysis oil comprises about 2 wt. % to about 30 wt. % of a C₁-C₄ hydrocarbon gas, about 10 wt. % to about 50 wt. % of a C₅-C₁₅ hydrocarbon oil, about 10 wt. % to about 40 wt. % of a wax, and about 1 wt. % to about 5 wt. % char. In certain embodiments, the pyrolysis oil comprises about 35 wt. % to about 75 wt. % of an olefin and/or a diolefin, about 10 wt. % to about 50 wt. % of a paraffin and/or an iso-paraffin, about 5 wt. % to about 25 wt. % of a naphthene, and about 5 wt. % to about 35 wt. % of an aromatic compound.

The method may further comprise heating the sample to a temperature from about 90° C. to about 200° C.

In some embodiments, the stream of air is passed at a flow rate from about 1 L/hour to about 20 L/hour.

In some embodiments, the volatile reaction product comprises an organic acid, a peroxide, and any combination thereof.

In certain embodiments, the sample excludes biodiesel fuel, a vegetable oil, diesel fuel, gasoline, and/or a lubricating oil.

In some embodiments, the pyrolysis oil further comprises a stabilizer, an antioxidant, a paraffin inhibitor, an asphaltene dispersant, a wax dispersant, a tar dispersant, a neutralizer, a surfactant, a biocide, a preservative, or any combination thereof.

The electrical conductivity of the measurement solution may be measured until an increase in electrical conductivity is detected.

The present disclosure also provides a method of determining the ability of an antioxidant to stabilize a pyrolysis oil. The method comprises heating a first sample comprising the pyrolysis oil to a first temperature from about 50° C. to about 220° C., passing a first stream of air through the sample of pyrolysis oil, forming a first volatile reaction product, transporting the first volatile reaction product into a first measurement solution comprising deionized water, and measuring an electrical conductivity of the first measurement solution for a first period of time until an increase in conductivity is detected. An antioxidant may then be added to a second sample of the pyrolysis oil and the method includes heating the second sample to the first temperature, passing a second stream of air through the second sample, forming a second volatile reaction product, transporting the second volatile reaction product into a second measurement solution comprising deionized water, measuring an electrical conductivity of the second measurement solution for a second period of time until an increase in conductivity is detected, and comparing the first period of time to the second period of time.

In some embodiments, the pyrolysis oil comprises about 2 wt. % to about 30 wt. % of a C₁-C₄ hydrocarbon gas, about 10 wt. % to about 50 wt. % of a C₅-C₁₅ hydrocarbon oil, about 10 wt. % to about 40 wt. % of a wax, and about 1 wt. % to about 5 wt. % char. In certain embodiments, the pyrolysis oil comprises about 35 wt. % to about 75 wt. % of an olefin and/or a diolefin, about 10 wt. % to about 50 wt. % of a paraffin and/or an iso-paraffin, about 5 wt. % to about 25 wt. % of a naphthene, and about 5 wt. % to about 35 wt. % of an aromatic compound.

The method may further comprise heating the first sample to a temperature from about 90° C. to about 200° C.

In some embodiments, the first and/or second stream of air is passed at a flow rate from about 1 L/hour to about 20 L/hour.

In some embodiments, the first volatile reaction product and/or the second volatile reaction product comprises an organic acid, a peroxide, and any combination thereof.

In certain embodiments, the first sample and the second sample exclude biodiesel fuel, a vegetable oil, diesel fuel, gasoline, and/or a lubricating oil.

In some embodiments, the pyrolysis oil further comprises a stabilizer, an additional antioxidant, a paraffin inhibitor, an asphaltene dispersant, a wax dispersant, a tar dispersant, a neutralizer, a surfactant, a biocide, a preservative, or any combination thereof.

In some embodiments, the antioxidant and/or additional antioxidant are independently selected from phenol-based antioxidants and/or amine-based antioxidants.

The present disclosure provides an additional method of determining the oxidative stability of a pyrolysis oil. The method comprises introducing a sample of the pyrolysis oil into a sample chamber, pressurizing the sample chamber to a pressure from about 200 kPa to about 700 kPa with O₂, heating the sample of the pyrolysis oil to a temperature from about 100° C. to about 180° C., monitoring a pressure within the sample chamber, and determining a time at which a decrease in the pressure within the sample chamber is detected.

In some embodiments, the pyrolysis oil comprises about 2 wt. % to about 30 wt. % of a C₁-C₄ hydrocarbon gas, about 10 wt. % to about 50 wt. % of a C₅-C₁₅ hydrocarbon oil, about 10 wt. % to about 40 wt. % of a wax, and about 1 wt. % to about 5 wt. % char. In some embodiments, the pyrolysis oil comprises about 35 wt. % to about 75 wt. % of an olefin and/or a diolefin, about 10 wt. % to about 50 wt. % of a paraffin and/or an iso-paraffin, about 5 wt. % to about 25 wt. % of a naphthene, and about 5 wt. % to about 35 wt. % of an aromatic compound.

In certain embodiments, the sample excludes biodiesel fuel, a vegetable oil, diesel fuel, gasoline, and/or a lubricating oil.

The present disclosure provides yet another method for determining the oxidative stability of a pyrolysis oil. The method comprises introducing a sample of the pyrolysis oil into a sample chamber, pressurizing the sample chamber to a pressure from about 100 psi to about 1,000 psi with O₂, heating the sample of the pyrolysis oil to a temperature of about 90° C. to about 250° C., holding the sample at the pressure and the temperature until an exothermic reaction is detected, and determining a period of time from a beginning of the pressurizing step to a time when the exothermic reaction is detected.

In some embodiments, the pyrolysis oil comprises about 2 wt. % to about 30 wt. % of a C₁-C₄ hydrocarbon gas, about 10 wt. % to about 50 wt. % of a C₅-C₁₅ hydrocarbon oil, about 10 wt. % to about 40 wt. % of a wax, and about 1 wt. % to about 5 wt. % char. In some embodiments, the pyrolysis oil comprises about 35 wt. % to about 75 wt. % of an olefin and/or a diolefin, about 10 wt. % to about 50 wt. % of a paraffin and/or an iso-paraffin, about 5 wt. % to about 25 wt. % of a naphthene, and about 5 wt. % to about 35 wt. % of an aromatic compound.

In certain embodiments, the sample excludes biodiesel fuel, a vegetable oil, diesel fuel, gasoline, and/or a lubricating oil.

The foregoing has outlined rather broadly the features and technical advantages of the present disclosure in order that the detailed description that follows may be better understood. Additional features and advantages of the disclosure will be described hereinafter that form the subject of the claims of this application. It should be appreciated by those skilled in the art that the conception and the specific embodiments disclosed may be readily utilized as a basis for modifying or designing other embodiments for carrying out the same purposes of the present disclosure. It should also be realized by those skilled in the art that such equivalent embodiments do not depart from the spirit and scope of the disclosure as set forth in the appended claims.

### BRIEF DESCRIPTION OF THE SEVERAL VIEWS OF THE DRAWINGS

A detailed description of the invention is hereafter described with specific reference being made to the drawings in which:

FIG. 1 shows certain components of a device that may be used to carry out the modified version of the Rancimat method disclosed herein.

### DETAILED DESCRIPTION

Unless otherwise defined, all technical and scientific terms used herein have the same meaning as commonly understood by one of ordinary skill in the art. In case of conflict, the present document, including definitions, will control. Examples of methods and materials are described below, although methods and materials similar or equivalent to those described herein can be used in practice or testing of the present disclosure. All publications, patent applications, patents and other reference materials mentioned herein are incorporated by reference in their entirety. The materials, methods, and examples disclosed herein are illustrative only and not intended to be limiting.

Unless otherwise indicated, an alkyl group as described herein alone or as part of another group is an optionally substituted linear or branched saturated monovalent hydrocarbon substituent containing from, for example, one to about sixty carbon atoms, such as one to about thirty carbon atoms, in the main chain. Examples of unsubstituted alkyl groups include methyl, ethyl, n-propyl, i-propyl, n-butyl, i-butyl, s-butyl, t-butyl, n-pentyl, i-pentyl, s-pentyl, t-pentyl, and the like.

The terms “aryl” or “ar” as used herein alone or as part of another group (e.g., arylene) denote optionally substituted homocyclic aromatic groups, such as monocyclic or bicyclic groups containing from about 6 to about 12 carbons in the ring portion, such as phenyl, biphenyl, naphthyl, substituted phenyl, substituted biphenyl or substituted naphthyl. The term “aryl” also includes heteroaryl functional groups. It is understood that the term “aryl” applies to cyclic substituents that are planar and comprise 4n+2n electrons, according to Huckel's Rule.

The term “substituted” as in “substituted alkyl,” means that in the group in question (i.e., the alkyl group), at least one hydrogen atom bound to a carbon atom is replaced with one or more substituent groups, such as hydroxy (—OH), alkylthio, phosphino, amido (—CON(R_{A})(R_{B}), wherein R_{A} and R_{B} are independently hydrogen, alkyl, or aryl), amino(—N(R_{A})(R_{B}), wherein R_{A} and R_{B} are independently hydrogen, alkyl, or aryl), halo (fluoro, chloro, bromo, or iodo), silyl, nitro (—NO₂), an ether (—OR_{A} wherein R_{A} is alkyl or aryl), an ester (—OC(O)R_{A} wherein R_{A} is alkyl or aryl), keto (—C(O)R_{A} wherein R_{A} is alkyl or aryl), heterocyclo, and the like.

When the term “substituted” introduces a list of possible substituted groups, it is intended that the term apply to every member of that group. That is, the phrase “optionally substituted alkyl or aryl” is to be interpreted as “optionally substituted alkyl or optionally substituted aryl.”

The terms “polymer,” “copolymer,” “polymerize,” “copolymerize,” and the like include not only polymers comprising two monomer residues and polymerization of two different monomers together, but also include (co)polymers comprising more than two monomer residues and polymerizing together more than two or more other monomers. For example, a polymer as disclosed herein includes a terpolymer, a tetrapolymer, polymers comprising more than four different monomers, as well as polymers comprising, consisting of, or consisting essentially of two different monomer residues. Additionally, a “polymer” as disclosed herein may also include a homopolymer, which is a polymer comprising a single type of monomer unit.

Unless specified differently, the polymers of the present disclosure may be linear, branched, crosslinked, structured, synthetic, semi-synthetic, natural, and/or functionally modified. A polymer of the present disclosure can be in the form of a solution, a dry powder, a liquid, or a dispersion, for example.

The term “antioxidant” as used herein is a compound that can inhibit, prevent or reduce oxidation, deterioration, degradation and gum formation. Antioxidants are capable of acting as scavengers preventing free radical formation or trapping free radicals when they form.

As used herein, the term “pyrolysis oil” refers to an oil derived from a recycled plastic feedstock. Processes to form the pyrolysis oil include breaking long-chain plastic polymers by thermochemical conversion at high temperatures, such as from about 400° C. to about 850° C., with limited or no oxygen and above atmospheric pressure. The resultant pyrolysis effluent is distilled and then condensed into pyrolysis oil.

The pyrolysis reaction produces a range of hydrocarbon products from gases (at temperatures from about 10° C. to about 50° C. and about 0.5 to about 1.5 atmospheric pressure and having 5 carbons or less); modest boiling point liquids (like gasoline (about 40 to about 200° C.) or diesel fuel (about 180 to about 360° C.)); a higher (e.g., about 250 to about 475° C.) boiling point liquid (oils and waxes), and some solid residues, commonly referred to as char.

Char is the material that is left once the pyrolytic process is complete and the pyrolysis oil is recovered. Char contains the additives and contaminants that enter the system as part of the feedstock. The char can be a powdery residue or substance that is more like sludge with a heavy oil component. Glass, metal, calcium carbonate/oxide, clay and carbon black are just a few of the contaminants and additives that will remain after the conversion process is complete and become part of the char.

Various plastic types, such as thermoplastic or thermoplastic waste, can be used in the above described process as the recycled plastic feedstock. The types of plastics commonly used include, but are not limited to, low-density polyethylene, high-density polyethylene, polypropylene, polystyrene, and combinations thereof.

The term “stabilizer” refers to a composition or compound that prevents or reduces discoloration of the pyrolysis oil, prevents or reduces the formation or settling out of insoluble products (e.g., gums) or combinations thereof.

The present disclosure provides novel test methods that may be used to measure oxidative stability of pyrolysis oils, differentiate internal antioxidant chemistries, evaluate the effectiveness of antioxidants in pyrolysis oils, predict the shelf-life of the pyrolysis oils, and determine concentration versus performance relationships.

In some embodiments, a modified Rancimat method may be used to achieve the foregoing goals. Although the Rancimat method (EN 14112) may be used to measure the oxidative stability of vegetable oils and/or biodiesel, it is not compatible with other types of oils, such as pyrolysis oils. Thus, the present inventors modified the method so that it could be used with pyrolysis oils.

In some embodiments, pyrolysis of the plastic results in pyrolysis oils that include about 2 to about 30 wt. % gas (C₁-C₄ hydrocarbon); about 10 to about 50 wt. % oil (C₅-C₁₅ hydrocarbon); about 10 to about 40 wt. % waxes (≥C₁₆ hydrocarbon); and about 1 to about 5 wt. % char.

The hydrocarbons that derive from the pyrolysis of waste plastic are a mixture of alkanes, alkenes, olefins and diolefins; the olefin group is generally between C₁ and C₂, viz. alpha-olefin, some alk-2-ene is also produced; the diene is generally in the alpha and omega position, viz. alk-α,ω-diene. In some embodiments, the pyrolysis of plastic produces paraffin compounds, isoparaffins, olefins, diolefins, naphthenes and aromatics. In some embodiments, the percentage of 1-olefins in the pyrolysis effluent is from about 25 to about 75 wt. %; or from about 35 to about 65 wt. %.

Depending on the processing conditions, the pyrolysis oil can have characteristics similar to crude oil from petroleum sources but may have varying amounts of olefins and diolefins. In some embodiments, the pyrolysis oil derived from waste plastic contains about 35 to about 65 wt. % olefins and/or diolefins, about 10 to about 50 wt. % paraffins and/or iso-paraffins, about 5 to about 25 wt. % naphthenes, and about 5 to about 35 wt. % aromatics. In some embodiments, the pyrolysis oil has about 15 to about 20 wt. % C₉-C₁₆; about 75 to about 87 wt. % C₁₆-C₂₉; and about 2 to about 5 wt. % C₃₀+, where the carbon chains are predominantly a mixture of alkanes, alkenes and diolefins. In other embodiments, the pyrolysis oil has about 10 wt. %<C₁₂, about 25 wt. % C₁₂-C₂₀, about 30 wt. % C₂₁-C₄₀ and about 35 wt. %>C₄₁, where the carbon chains are predominantly a mixture of alkanes, alkenes and diolefins.

In some embodiments, the pyrolysis oil has a range of alpha or omega olefins monomer constituents which can precipitate from the oil at a temperature greater than is desired or intended during storage, use, or transport. In some embodiments, the pyrolysis oil is about 25 to about 75 wt. % olefins and/or diolefins; about 35 to about 75 wt. % olefins and/or diolefins; about 45 to about 75 wt. % olefins and/or diolefins; or about 55 to about 75 wt. % olefins and/or diolefins.

In some embodiments, the samples of pyrolysis oils tested in accordance with the present disclosure exclude biodiesel fuel, diesel fuel, gasoline, animal fats, and/or vegetable oils.

The inventors studied and experimented with various air flow rates, test temperatures, and sample sizes. Finding an appropriate set of these test method parameters was vital to allow the modified Rancimat method to be applied to pyrolysis oils.

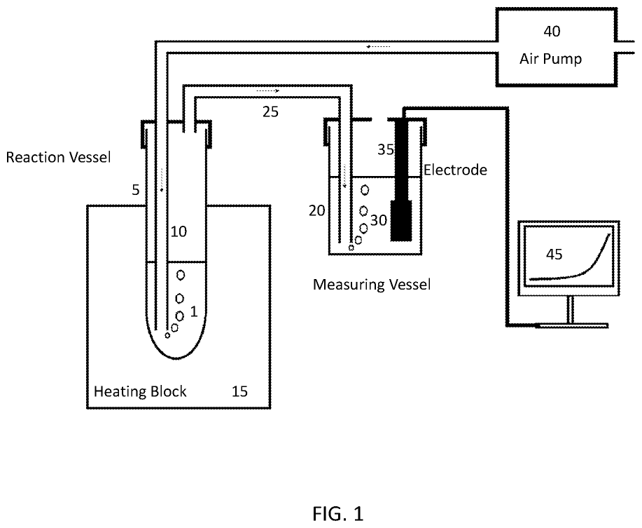

According to the modified version of the Rancimat method discovered by the inventors, and as depicted in FIG. 1, a sample of pyrolysis oil (1) is added to a reaction vessel (5), which includes an air inlet tube (10) immersed in the sample. The reaction vessel (5) is sealed by a cap, lid, or similar device and a stream of air originating from an air pump (40) is passed through the pyrolysis oil sample (1) in the reaction vessel (5) via the air inlet tube (10). During the process, the reaction vessel (5) is heated to a specified temperature. In some embodiments, the reaction vessel (5) is placed on top of a heating block (15) or hot plate. The temperature may be held constant throughout the test.

These isothermal thermo-oxidative conditions result in the oxidation of the sample (1). Volatile reaction/oxidation products are formed, which are transported into a measurement vessel (20) by a conduit (25) containing the airstream and absorbed into the measurement solution (30), which comprises, consists of, or consists essentially of deionized water.

The reaction products may include, for example, fatty acids, such as fatty acid methyl esters, peroxides, volatile organic compounds, and low molecular weight organic acids, such as formic acid and/or acetic acid.

In some embodiments, the measurement vessel (20) contains from about 10 mL to about 100 mL of deionized water, such as about 20 mL, about 30 mL, about 40 mL, about 50 mL, about 60 mL, about 70 mL, about 80 mL, or about 90 mL of deionized water.

The measurement vessel (20) also includes an electrode (35) immersed in the measurement solution (30). The type of electrode (35) is not particularly limited, so long as it can measure conductivity of an aqueous solution. The electrical conductivity of the measurement solution (30) increases due to the absorption of the reaction/oxidation products, such as volatile carboxylic acids. The time until a significant increase in the conductivity occurs is called induction time. Oxidative stability is expressed as “induction time,” and longer induction times correspond to higher oxidative stability. Any component of the device, such as the electrode (35), may be in communication with and transmit data to a computer (45) or central processing unit.

The inventors determined that an appropriate air flow rate may comprise, in some embodiments, a rate of about 1 L/hour to about 20 L/hour. For example, an appropriate air flow rate may include from about 5 L/hour to about 15 L/hour, such as about 6 L/hour, about 7 L/hour, about 8 L/hour, about 9 L/hour, about 10 L/hour, about 11 L/hour, about 12 L/hour, about 13 L/hour, or about 14 L/hour.

In some embodiments, the sample (1) may be heated to a temperature ranging from about 50° C. to about 220° C. For example, the sample may be heated to a temperature ranging from about 60° C. to about 220° C., about 70° C. to about 220° C., about 80° C. to about 220° C., about 90° C. to about 220° C., about 100° C. to about 220° C., about 120° C. to about 220° C., about 140° C. to about 220° C., about 160° C. to about 220° C., about 180° C. to about 220° C., or about 200° C. to about 220° C.

Any commercially available device that can conduct a test according to the Rancimat method may be used in accordance with the present disclosure, such as the 873 Biodiesel Rancimat from Metrohm or the 743 Rancimat from Metrohm, so long as the device can at least measure conductivity and accommodate the temperatures, sample sizes, and air flow rates needed to carry out the presently disclosed methods.

Pyrolysis oils that are liquid at room temperature may be added directly to the reaction vessel for testing. A pyrolysis oil that is a solid or semi-solid at room temperature can be melted and then added to the reaction vessel.

Thermo-oxidative stability is one of the most important properties of pyrolysis oils, and high oxidation stability means longer shelf-life and utility-life. Pyrolysis oils as produced by their manufacturers may not have sufficient oxidation stability to support the performance requirements. Improved oxidative stability can therefore be obtained by the incorporation of an antioxidant product that functions by interaction with the free radicals produced during the process of oxidation. Due to the differences in their inherent oxidative stability, different pyrolysis oils may respond differently to antioxidants and need to be investigated thoroughly.

The stability of the pyrolysis oil can be improved by additives that inhibit, prevent or reduce gum formation, discoloration and oxidation. In some embodiments, stability is achieved through the use of an antioxidant.

In some embodiments, the pyrolysis oil may further comprise a stabilizer, an antioxidant, a paraffin inhibitor, an asphaltene dispersant, a wax dispersant, a tar dispersant, a neutralizer, a surfactant, a biocide, a preservative, or any combination thereof.

The present disclosure provides methods for determining the effectiveness of a particular antioxidant in a particular pyrolysis oil. The amount of olefin content varies in different types of pyrolysis oils so certain antioxidants will work better than others. Higher contents of olefin may lead to less oxidative stability.

In some embodiments, a method for determining the ability of an antioxidant to stabilize a pyrolysis oil is provided, which includes heating a first sample comprising the pyrolysis oil to a first temperature from about 50° C. to about 220° C., passing a first stream of air through the sample of pyrolysis oil, forming a first volatile reaction product, transporting the first volatile reaction product into a first measurement solution comprising deionized water, and measuring an electrical conductivity of the first measurement solution for a first period of time until an increase, such as a significant increase, in conductivity is detected.

The conductivity of any measurement solution provided herein may be continuously or intermittently measured throughout the tests. There may be minor conductivity modifications detected throughout the test but the induction time is generally measured from the time at which the test starts to the time at which a significant increase in conductivity is detected. In terms of conductivity, a “significant increase” may include an increase of about 10% or more, such as about 20% or more, about 30% or more, about 40% or more, or about 50% or more. For example, a “significant increase” may be from about 10% to about 1,000%, from about 20% to about 1,000%, from about 30% to about 1,000%, from about 40% to about 1,000%, or from about 50% to about 1,000%.

A second sample of the same pyrolysis oil may be taken and added to a reaction vessel. A particular antioxidant may also be added to the second sample/reaction vessel. The second sample is then heated to the first temperature and a second stream of air is passed through the second sample at a flow rate which is the same or substantially similar to the flow rate used with the first sample. Volatile reaction products are once again formed and transported by the air flow into a second measurement solution comprising deionized water.

Electrical conductivity of the second measurement solution is monitored throughout the test until a significant increase in conductivity is detected. The time period from the beginning of the second test to the point at which a significant increase in conductivity was detected may be referred to as the second period of time.

The first period of time is then compared to the second period of time to determine the effectiveness of the particular antioxidant. For example, if the first period of time was about two hours and the second period of time was about four hours, then the antioxidant effectively increased the induction time of the particular pyrolysis oil.

The antioxidants encompassed by the present disclosure are not limited and may include, for example, phenol-based antioxidants and/or amine-based antioxidants. The antioxidants work against, for example, ethylenically unsaturated monomers to reduce contamination, which in turn inhibits, prevents or reduces gum formation, discoloration, or both of the synthetic feedstock.

Examples of antioxidants include phenolic antioxidants, such as hindered phenols and phenylenediamines thereof, to prevent oxidation and unwanted polymerization (e.g., radical) of ethylenically unsaturated monomers.

In some embodiments, the phenolic antioxidant is a hindered phenol. In some embodiments, hindered phenol is an alkylated phenolic antioxidant. In some embodiments, the antioxidant is a hindered phenol including alkyl-substituted hindered phenols, aromatic amines, or mixtures and combinations thereof. In some embodiments, the phenol is a butyl substituted phenol containing 2 or 3 t-butyl groups.

In some embodiments, the hindered phenols are generally alkyl phenols of the formula:

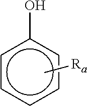

wherein Rₐ is independently an alkyl group containing from 1 up to about 24 carbon atoms and a is an integer of from 1 up to 5, 1 to 4, 1 to 3 or 1 to 2. In some embodiments, Rₐ contains from 4 to 18 carbon atoms, or from 4 to 12 carbon atoms. Rₐ may be either straight chained or branched chained. In some embodiments, the hindered phenolic antioxidant is an alkyl phenol selected from ter-butyl, OH, OCH₃ methylphenyl or mixtures thereof.

In some embodiments, the hindered phenol is 2-tert-butylphenol, 4-tert-butylphenol 2,4-di-tert-butylphenol, 2,6-di-tert-butylphenol, 2,4, 6-tri-tert-butylphenol, 2,6-di-tert-butyl-4-methylphenol, 2-tert-butyl-4,6-dimethylphenol, 2,6-di-tert-butyl-4-ethylphenol, 2,6-di-tert-butyl-4-n-butylphenol, 2,6-di-tert-butyl-4-i-butylphenol, 2,6-dicyclopentyl-4-methylphenol, 2-(α-methylcyclohexyl)-4,6-dimethylphenol, 2,6-dioctadecyl-4-methylphenol, 2,4,6-tricyclohexylphenol, 2,6-di-tert-butyl-4-methoxymethylphenol, 2,6-dinonyl-4-methylphenol, 2,6-di-tert-butyl-4-methoxyphenol, 2,5-di-tert-butylhydroquinone, 2,5-di-tert-amylhydroquinone, 2,6-diphenyl-4octadecyloxyphenol, 2,2′-thiobis(6-tert-butyl-4-methylphenol), 2,2′-thiobis(4-octylphenol), 4,4′-thiobis(6-tert-butyl-3-methylphenol), 4,4′-thiobis(6-tert-butyl-2-methylphenol), 2,2′-methylenebis(6-tert-butyl-4-methylphenol), 2,2′-methylenebis(6-tert-butyl-4-ethylphenol), 2,2′-methylenebis 4-methyl-6-(α-methylcyclohexyl)pheno!, 2,2′-methylenebis(4-methyl-6-cyclohexylphenol), 2,2′-methylenebis(6-nonyl-4-methylphenol), 2,2′-methylenebis(4,6-di-tert-butylphenol), 2,2′-ethylidenebis(4,6-di-tert-butylphenol), 2,2′-ethylidenebis(6-tert-butyl-4-isobutylphenol), 2,2′-methylenebis 6-(α-methylbenzyl)-4-nonylphenol, 2,2′-methylenebis 6-(α,α-dimethylbenzyI)-4-nonylphenol, 4,4′-methylenebis(2,6-di-tert-butylphenol), 4,4′-methylenebis(6-tert-butyl-2-methylphenol), 1,1-bis(5-tert-butyl-4-hydroxy-2-methylphenyl)butane, 2,6-bis(3-tert-butyl-5-methyl-2-hydroxybenzyl)-4-m ethylphenol, 1,1, 3-tris(5-tert-butyl-4-hydroxy-2-m ethylphenyl)butane, 1,1-bis(5-tert-butyl-4-hydroxy-2-methylphenyl)-3-n-dodecylmercaptobutane, ethylene glycol bis 3,3-bis(3′-tert-butyl-4′-hydroxyphenyl)butyrate!, bis(3-tert-butyl-4-hydroxy-5-methylphenyl)dicyclopentadiene, bis 2-(3′-tert-butyl-2′-hydroxy-5′-methylbenzyl)-6-tert-butyl-4-methylphenylterephthalate, 1,3,5-tris(3,5-di-tert-butyl-4-hydroxybenzyl)-2,4,6-trimethylbenzene, bis(3,5-di-tert-butyl-4-hydroxybenzyl)sulfide, isooctyl 3,5-di-tert-butyl-4-hydroxybenzylmercaptoacetate, bis(4-tert-butyl-3-hydroxy-2,6-dimethylbenzyl) dithioterephthalate, 1,3,5-tris(3,5-di-tert-butyl-4-hydroxybenzyl) isocyanurate, 1,3,5-tris(4-tert-butyl-3-hydroxy-2,6-dimethylbenzyl)isocyanurate, dioctadecyl 3,5-di-tert-butyl-4-hydroxybenzylphosphonate and the calcium salt of monoethyl 3,5-di-tert-butyl-4-hydroxybenzylphosphonate, a tert-butylcatechol, or any combination thereof.

In some embodiments, the antioxidant is an aromatic amine. In some embodiments, the antioxidant is an alkylated phenylenediamine, which can include an unsubstituted phenylenediamine, N-substituted phenylenediamine or N,N′-substituted phenylenediamine targeted towards an ethylenically unsaturated monomer, and any combination thereof. Examples of phenylenediamine are 1,4-phenylenediamine, N,N′-dimethyl-p-phenylenediamine, N, N′-di-sec-butyl-p-phenylenediamine, N, N′-di-sec-butyl-1,4-phenylenediamine, N-phenyl-N′-dibutyl-p-phenylenediamine, N-phenyl-N′-(1,4-dimethylphenyl)-p-phenylenediamine, N-phenyl-N′-(1,3-dimethylbutyl)-p-phenylenediamine, and any combination thereof. Phenylenediamines can also include p- or m-phenylenediamine itself (PDA); N,N′-diphenyl-p-phenylenediamine; N,N,N′,N′-tetramethyl-p-phenylenediamine; N,N′-bis-(1,4-dimethylpentyl)-phenylenediamine; N-phenyl-N′-(1,4-dimethylpentyl) p-phenylenediamine; N-phenyl-N′-(1,3-dimethylbutyl) p-phenylenediamine; N-phenyl-N-cyclohexyl p-phenylenediamine; N, N′-dinaphthyl p-phenylenediamine; N-isopropyl-N′-phenyl p-phenylenediamine; N-aminoalkyl-N′-phenyl p-phenylenediamine; N-(2-methyl-2-aminopropyl)-N′-phenyl p-phenylenediamine; phenyl-b-isopropyl-aminophenylamine; p-hydroxydiphenylamine; p-hydroxylphenyl-b-naphthylamine; 1,8-naphthalenediamine, and any combination thereof.

Hindered phenolic compounds can include o- and p-sec-butylphenol; 2,4-di-sec-butylphenol; 2,6-di-sec-butylphenol; 2,4,6-tri-sec-butylphenol; 2,4,6-trimethylphenol; butylated hydroxytoluene (BHT, also known as 2,6-tert-butyl-4-methylphenol and 2,6-tert-butyl p-cresol); 2,6-dibutyl-4-methylphenol; hydroquinone; monomethylether of hydroquinone (MEHQ); 2,6-bis (1,6 dimethylethyl-4-(1-methylpropyl) phenol), b-naphthoquinone; N-phenyl p-aminophenol; and combinations thereof. In some embodiments, the antioxidant comprises 2-tert-butylphenol, 4-tert-butylphenol 2,4-di-tert-butylphenol, 2,6-di-tert-butylphenol, 2,4, 6-tri-tert-butylphenol, 1, 2, 4-trimethyl benzene, N, N′-di-sec-butyl-1,4-phenylenediamine or combinations thereof.

In some embodiments, the antioxidant may be diluted in a solvent, such as heavy aromatic naphtha, kerosene, toluene, ethylbenzene, isomeric hexanes, and mixtures thereof, for example.

While the amount of antioxidant added to the pyrolysis oil depends on a number of factors, such as the type of pyrolysis oil, the type of contamination, and the local operating conditions, examples of amounts introduced into the pyrolysis oil include from about 1 ppm to about 500 ppm, such as from about 5 ppm to about 500 ppm, about 10 ppm to about 500 ppm, about 20 ppm to about 500 ppm, about 30 ppm to about 500 ppm, about 40 ppm to about 500 ppm, about 50 ppm to about 500 ppm, about 60 ppm to about 500 ppm, about 70 ppm to about 500 ppm, about 80 ppm to about 500 ppm, about 90 ppm to about 500 ppm, about 100 ppm to about 500 ppm, about 5 ppm to about 450 ppm, about 5 ppm to about 400 ppm, about 5 ppm to about 350 ppm, about 5 ppm to about 300 ppm, about 5 ppm to about 250 ppm, about 5 ppm to about 200 ppm, about 5 ppm to about 150 ppm, about 5 ppm to about 100 ppm, about 10 ppm to about 300 ppm, about 10 ppm to about 250 ppm, about 50 ppm to about 250 ppm, or about 50 ppm to about 200 ppm.

To evaluate the oxidative stability of a pyrolysis oil, the present disclosure provides various methods in addition to the modified Rancimat method. For example, the present inventors determined that the rapid small scale oxidation test (RSSOT), as described in ASTM D7545, can be used. The RSSOT test was developed for characterizing the stability of middle distillate fuels, such as diesel fuel, heating oil, and biodiesel, but the inventors found that it can also be used to characterize the oxidative stability of pyrolysis oils, despite the significant compositional and property differences between pyrolysis oils and the aforementioned fuels for which the test was designed.

Details of the test apparatus and method are described in ASTM D7545, which is expressly incorporated by reference herein. In general, a sample of pyrolysis oil is introduced to a sample chamber, which is then pressurized to a pressure from about 200 kPa to about 700 kPa with O₂ and then heated from about 100° C. to about 180° C.

In some embodiments, the pressure is from about 200 kPa to about 700 kPa, such as from about 300 kPa to about 700 kPa or about 500 kPa to about 700 kPa.

In some embodiments, the sample is heated from about 100° C. to about 160° C., such as from about 100° C. to about 140° C., about 100° C. to about 120° C., about 130° C. to about 180° C., or about 150° C. to about 180° C.

Pressure of the sample chamber is monitored over time and the time at which the pressure begins to drop due to oxygen consumption (referred to as the breakpoint of the sample, reported in units of time) provides an indication of the sample's oxidative stability.

In some embodiments, the present disclosure provides a method of determining the oxidative stability of a pyrolysis oil. The method comprises introducing a sample of the pyrolysis oil into a sample chamber. The pyrolysis oil may be any of the pyrolysis oils disclosed in or contemplated by the present disclosure. The sample chamber/sample may be pressurized to a pressure of about 700 kPa with O₂. The sample chamber/sample is also heated to a temperature of about 140° C. While heating and pressurizing, the pressure within the sample chamber is monitored over time. The time at which the pressure begins to drop due to oxygen consumption indicates the oxidative stability of the sample.

The present inventors also determined that a pressurized differential scanning calorimetry (PDSC) test may be used evaluate the oxidative stability of a pyrolysis oil. The PDSC method (ASTM D6186, incorporated herein by reference) is a technique used to determine the oxidative stability of lubricant oils. The present inventors postulated that the PDSC oxidation test method can be modified and also be employed for waste-plastics derived feedstocks, such as pyrolysis oils.

In accordance with the PDSC method, a sample of pyrolysis oil is heated to a temperature (e.g., about 160° C.) under increased oxygen pressure (e.g., about 500 psi). Once the target temperature is achieved, the sample remains subjected to the thermo-oxidative conditions in isothermal mode until the oxidation begins. The onset of exothermic oxidation is called the oxidation induction time (OIT). A higher OIT indicates a higher oxidative stability.

In some embodiments, the present disclosure provides a method of determining the oxidative stability of a pyrolysis oil. The method comprises introducing a sample of the pyrolysis oil into a sample chamber, pressurizing the sample chamber to a pressure of about 100 psi to about 1,000 psi with O₂, and heating the sample of the pyrolysis oil to a temperature of about 90° C. to about 250° C. The sample may be held at the elevated pressure and temperature until an exothermic reaction is detected.

In some embodiments, the pressure is between about 200 psi and about 1,000 psi, such as from about 200 psi to about 800 psi, from about 300 psi to about 600 psi, or from about 400 psi to about 500 psi. The time from when pressurization begins to the time at which the exothermic reaction is detected is determined and provides an indication of the oxidative stability of the pyrolysis oil.

In some embodiments, the temperature is between about 100° C. to about 250° C., about 120° C. to about 250° C., about 140° C. to about 250° C., about 160° C. to about 250° C., about 180° C. to about 250° C., about 200° C. to about 250° C., about 220° C. to about 250° C., about 90° C. to about 200° C., about 90° C. to about 150° C., or about 90° C. to about 120° C.

The PDSC method offers certain advantages, such as a short testing period (typically only a few minutes to conduct the experiment) and a small sample size (typically only a few mg).

The foregoing may be better understood by reference to the following examples, which are intended for illustrative purposes and are not intended to limit the scope of the disclosure or its application in any way.

### EXAMPLES

With regard to the modified Rancimat method disclosed herein, to understand the relationship between test temperature and induction period, oxidation stability of untreated and an antioxidant (Stabilization Additive 3) treated pyrolysis oil was measured at a range of temperatures. Data is provided below in Table 1. A relationship between the induction period (rate of oxidation) and temperature change, with a strong exponential relationship for the curves, was observed. When developing a test method for any specific pyrolysis oil, this allows the user to predict and target induction period value and optimize test procedure.

TABLE 1

| | IP (h), 5 g sample, 10 L/h air flow | |
|---|---|---|
| T | | Stabilization Additive 3 |
| (° C.) | Blank | (100 ppm) |
| 140 | 9.11 | 15.33 |
| 150 | 5.23 | 8.37 |
| 160 | 2.26 | 3.31 |
| 170 | 1.48 | 2.15 |

The effect of the sample size on the test result was a variable that the inventors investigated. To evaluate this, oxidation stability of a pyrolysis oil was determined with different sample quantities in reaction vessels at temperatures ranging from about 160° C. to about 170° C. Results are shown in Table 2. Sample amounts yielded constant induction times at about 160° C. Under those conditions, the inventors observed some evaporation at a sample size of about 7.5 g at about 170° C. after about 60 minutes. Typically, a small reduction of the sample's volume during the procedure would induce a slight acceleration of the oxidation process and shorten the OSI (oxidation stability index) \ IP (induction period) measurements to a certain degree.

TABLE 2

| Sample | IP (h), | IP (h), |
|---|---|---|
| weight (g) | 160° C. | 170° C. |
| 7.5 | 4.75 | 2.79 |
| 10 | 4.64 | 3.15 |
| 12.5 | 4.71 | 3.16 |

In another set of experiments, samples of pyrolysis oils were obtained and subjected to accelerated thermo-oxidative conditions under a series of testing protocols by modifying either the temperature, the air flow or the sample size. By varying the test parameters, alterations in the relative oxidative characteristics of the samples were determined. The modified Rancimat results (Induction Period) presented in Table 3 below are for pyrolysis oil samples obtained from various suppliers.

TABLE 3

| Sample | | | | | | | | |
|---|---|---|---|---|---|---|---|---|
| Description of | Induction Period (hours) at a given Temperature | | | | | | | |
| Pyrolysis Oil | 110° C. | 120° C. | 130° C. | 140° C. | 150° C. | 160° C. | 170° C. | 180° C. |
| Sample 1 | 0.28 | | 0.19 | | 0.10 | | | |
| Sample 2 | 0.47 | | 0.27 | | 0.15 | | | |
| Sample 3 | 0.55 | | 0.34 | | | | | |
| Sample 4 | | | 0.31 | 0.23 | | | | |
| Sample 5 | 1.00 | 0.76 | 0.68 | | | | | |
| Sample 6 | | | 5.22 | 1.92 | 0.88 | | | |
| Sample 7 | | | 3.95 | 1.66 | 0.98 | | | |
| Sample 8 | | | | 2.71 | 1.05 | | | |
| Sample 9 | | | | 3.28 | 1.92 | 1.34 | | |
| Sample 10 | | | | | 4.03 | 2.57 | 1.13 | |
| Sample 11 | | | | 9.83 | 4.59 | 2.26 | 1.48 | 1.11 |
| Sample 12 | | | | | 4.81 | | | 0.52 |
| Sample 13 | | | | | 7.08 | | | |
| Sample 14 | | | | | 7.39 | | | 1.03 |
| Sample 15 | | | | | 10.92 | 6.14 | 2.67 | 1.77 |

These results clearly demonstrate that by selecting an appropriate set of parameters, the disclosed test methods can be used to measure the thermo-oxidative stability of a wide range of pyrolysis oils, differentiate between pyrolysis oils of various levels of oxidation stability, and demonstrate that the extraction process removes the natural antioxidant present in the byproduct formation and hence the induction time is decreased for the extracted version. Prior art methods cannot differentiate the antioxidant performance in high byproduct forming pyrolysate oils.

The data below show that the disclosed method can be conveniently used to evaluate various antioxidants, to discriminate between antioxidant additives, and determine their optimum treat-rate for a wide range of pyrolysis oils. Data in Table 4 below shows how three different incumbent antioxidant products perform at different treat-rates in a pyrolysis oil in the modified Rancimat test at about 150° C. with about 10 L/h air flow.

TABLE 4

| Sample Description | IP (h) | |
|---|---|---|
| Antioxidant | 500 ppm | 250 ppm |
| Blank (No AO) | | |
| 2,6-di-tert-butylphenol | 11.64 | 8.02 |
| Stabilization Additive 2 | 19.60 | 13.28 |
| Stabilization Additive 3 | 15.25 | 10.94 |
| Stabilization Additive 1 | 14.12 | 9.69 |
| Irganox ® L-57 | 4.66 | 4.54 |
| t-butylcatechol (50%) | 7.33 | 6.60 |

Data in Table 5 below shows how three different antioxidant products perform at different treat-rates in a pyrolysis oil in the modified Rancimat test at about 150° C. with about 10 L/h air flow.

TABLE 5

| | IP (h) | | |
|---|---|---|---|
| Conc | Stabilization | Stabilization | Stabilization |
| (ppm) | Additive 2 | Additive 3 | Additive 1 |
| 0 | 4.97 | 5.23 | 4.97 |
| 50 | 6.25 | 6.75 | 6.63 |
| 100 | 11.31 | 8.37 | 7.34 |
| 250 | 13.28 | 10.94 | 9.69 |
| 500 | 29.31 | 15.25 | 14.12 |

In the following examples, the effect of stabilization/antioxidant additives to enhance the thermo-oxidative stability of a wide range of pyrolysis oils (obtained from different suppliers) was quantitatively demonstrated by a modified version of the Rancimat method.

According to the modified version of the Rancimat method carried out by the inventors, a stream of purified air is passed through the pyrolysis oil sample, which is heated to a specified temperature. These isothermal thermo-oxidative conditions result in the oxidation of the sample. Volatile reaction/oxidation products are formed, which are transported into the measuring vessel by the airstream and absorbed into the measuring solution (deionized water). The electrical conductivity of the measuring solution increases due to the absorption of the reaction/oxidation products. The time until a sharp increase in the conductivity occurs is called induction time. Oxidative stability is expressed as “induction time,” and longer induction times correspond to higher oxidative stability.

The following example shows the relative efficacy of various antioxidants at a treat rate of about 500 ppm in improving the thermo-oxidative stability of a pyrolysis oil. The induction time (IT) determinations were made at about 150° C. and about 10 L/h of air.

TABLE 6

| | Induction time |
|---|---|
| | (h) measured at |
| Sample Description | 150° C. |
| Blank (No AO)—Supplier 1 | 4.47 |
| 2,6-Di-tert-butylphenol | 11.64 |
| Stabilization Additive 1 | 14.12 |
| Stabilization Additive 2 | 15.25 |
| Stabilization Additive 3 | 19.60 |
| 2,6-Di-t-butyl-4- | 5.40 |
| (dimethylaminomethyl)phenol | |
| Irganox ® L-57 | 4.66 |
| t-Butylcatechol | 7.33 |

Among the tested antioxidants, the Stabilization Additive 3 was the best performer whereas Irganox® L-57 was the worst performer as it provided an insignificant increase in induction time compared to the untreated pyrolysis oil.

Stabilization Additive 1 is defined as follows:

| Component | Wt % |
|---|---|
| 2,6-di-tert-butylphenol | 61-80 |
| Heavy aromatic naptha | 14-17 |
| 2,4-di-tert-butylphenol | 8-12 |
| 2,4,6-tri-tert-butylphenol | 5-8 |
| Naphthalene | 1-3 |
| 2-tert-butylphenol | 1-2 |
| 1,2,4-trimethylbenzene | 1-2 |
| Ethylbenzene | 0.0001-0.0002 |

Stabilization Additive 2 is defined as follows:

| Component | Wt % |
|---|---|
| N, N'-di-sec- | 50-60 |
| butylphenylenediamine | |
| 2,6-di-tert-butylphenol | 35-40 |
| 2,4-di-tert-butylphenol | 3-8 |
| 2,4,6-tri-tert-butylphenol | 3-6 |
| 2-tert-butylphenol | 0.5-1.5 |

Stabilization Additive 3 is defined as follows:

| Component | Wt % |
|---|---|
| N, N'-di-sec- | 50 |
| butylphenylenediamine | |
| Kerosene | 50 |

The following examples demonstrate that the thermo-oxidative stability of pyrolysis oils can be improved to different extents by increasing the concentration of an antioxidant additive. The results are provided in the tables provided below. In a first experiment, the effect of concentration of Stabilization Additive 1 on the performance at about 140° C., about 10 L/h air for a pyrolysis oil from Supplier 2 was evaluated.

TABLE 7

| | | Induction time |
|---|---|---|
| | Conc. | (h) measured at |
| Sample Description | (ppm) | 140° C. |
| Blank (No AO)— | 0 | 2.28 |
| Supplier 2 | | |
| Stabilization Additive 1 | 250 | 3.91 |
| Stabilization Additive 1 | 500 | 6.60 |
| Stabilization Additive 1 | 1000 | 12.6 |

In a second experiment, the effect of concentration of Stabilization Additive 2 on the performance at about 150° C., about 10 L/h air for a pyrolysis oil from Supplier 3 was evaluated.

TABLE 8

| | | Induction time |
|---|---|---|
| | Conc. | (h) measured at |
| Sample Description | (ppm) | 150° C. |
| Blank (No AO)— | 0 | 4.97 |
| Supplier 3 | | |
| Stabilization Additive 2 | 100 | 8.37 |
| Stabilization Additive 2 | 250 | 10.94 |
| Stabilization Additive 2 | 500 | 15.25 |

The following example demonstrates the effect of stabilization/antioxidant additives in substantially improving the thermo-oxidative stability of a pyrolysis oil having very poor baseline oxidative stability. The results are provided in the table below. This pyrolysis oil, without any antioxidant added to it, had poor stability as indicated by a very low OSI value (about 0.31 h at about 130° C., about 10 L/h air).

TABLE 9
Performance of different antioxidants at about
130° C. for a pyrolysis oil from Supplier 4

| | | Induction time |
|---|---|---|
| | Conc. | (h) measured at |
| Sample Description | (ppm) | 130° C. |
| Blank (Untreated)— | 0 | 0.31 |
| Supplier 4 | | |
| Stabilization Additive 1 | 500 | 0.72 |
| Stabilization Additive 2 | 500 | 2.71 |
| Stabilization Additive 3 | 500 | 3.17 |

Of the tested additives, Stabilization Additive 2 and Stabilization Additive 3, at a treat rate of about 500 ppm, caused a significant increase (9-10 times) in the induction time for this feedstock.

The following example shows the relative efficacy of antioxidants in improving the thermo-oxidative stability of a waxy plastics pyrolysate. This waxy pyrolysate (which was a solid at room temperature) was first melted at about 80° C. and then treated with different antioxidant additives. The induction time (IT) determinations were made at about 150° C. and about 10 L/h of air.

TABLE 10
Performance of different antioxidants at about
150° C. for a waxy pyrolysate from Supplier 5

| | IT (h) measured | |
|---|---|---|
| Sample | at 150° C. | |
| Description | 250 ppm | 500 ppm |
| Blank (Untreated)— | 7.12 | 7.12 |
| Supplier 5 | | |
| Stabilization Additive 1 | 8.78 | 10.42 |
| Stabilization Additive 2 | 14.63 | 21.38 |
| Stabilization Additive 3 | 14.56 | 20.85 |

At a treat rate of about 250 ppm, both Stabilization Additive 2 and Stabilization Additive 3 doubled the induction time, when compared with untreated pyrolysis oil. At a treat rate of about 500 ppm, these two additives increased the induction time by almost three times.

An additional experiment was carried out in connection with the RSSOT method. Details of the test apparatus and method are described in ASTM D7545. Generally, samples of pyrolysis oil were introduced to a sample chamber, which was pressurized to about 700 kPa with O₂ and heated to about 140° C. Pressure of the sample chamber was monitored over time and the time at which the pressure began to drop due to oxygen consumption (referred to as the breakpoint of the sample, reported in units of time) provided an indication of the sample's oxidative stability.

Below are two examples of how this method can be used to characterize the oxidative stability of pyrolysis oils. Each of the pyrolysis oils was received from a different supplier and was produced via distinct processes and feedstock sources. In the first example (AA), a significant increase in the induction period was observed as antioxidant concentration was increased, indicating both the efficacy of the antioxidant at improving stability and the utility of the RSSOT method in quantifying the samples' stability.

In the second example (BB), the untreated sample exhibited a shorter induction period (relative to Example AA), indicating this pyrolysis oil had a lower oxidative stability than the untreated oil in the first example. Likewise, the extent of improvement upon treatment with antioxidant was reduced compared to the first example. Regardless of sample stability, however, this example provides further support for use of the RSSOT method in characterizing the oxidative stability of both treated and untreated pyrolysis oils.

### Example AA

| | Induction |
|---|---|
| Sample | Period (min) |
| Untreated pyrolysis oil | 28 |
| (blank) | |
| 250 ppm Stabilization | 49 |
| Additive 1 | |
| 500 ppm Stabilization | 60 |
| Additive 1 | |
| 1000 ppm Stabilization | 86 |
| Additive 1 | |

### Example BB

| | Induction |
|---|---|
| Sample | Period (min) |
| Untreated pyrolysis oil | 20 |
| (blank) | |
| 500 ppm Stabilization | 25 |
| Additive 1 | |

Finally, an example demonstrating the applicability of the PDSC method for pyrolysis oils is provided below. The samples included:
blank pyrolysis oil (no AO)
pyrolysis oil treated with 250 ppm Stabilization Additive 2
pyrolysis oil treated with 250 ppm Stabilization Additive 3
pyrolysis oil treated with 250 ppm Stabilization Additive 1

The test parameters were as follows:
Instrument: DSC25P
Isothermal Temp: about 160° C.
Purge gas: Oxygen (about 500 PSI)
Pan type: Tzero Pan
Sample Mass: about 3 mg
Ramp about 10° C./min to about 160° C.

OIT data for four samples is shown below in Table 11.

TABLE 11

| | OIT @, 160° C., |
|---|---|
| Sample | onset time (min) |
| Pyrolysis oil with no AO | 27.68 |
| Pyrolysis oil treated with 250 ppm | 44.07 |
| Stabilization Additive 2 | |
| Pyrolysis oil treated with 250 ppm | 40.34 |
| Stabilization Additive 3 | |
| Pyrolysis oil treated with 250 ppm | 31.88 |
| Stabilization Additive 1 | |

This data shows that PDSC can be used to determine the oxidative stability of pyrolysis oils and discern the efficacy of different antioxidant additives (as well as their concentration) for pyrolysis oils.

All of the compositions and methods disclosed and claimed herein can be made and executed without undue experimentation in light of the present disclosure. While this invention may be embodied in many different forms, there are described in detail herein specific preferred embodiments of the invention. The present disclosure is an exemplification of the principles of the invention and is not intended to limit the invention to the particular embodiments illustrated. In addition, unless expressly stated to the contrary, use of the term “a” is intended to include “at least one” or “one or more.” For example, “a stabilizer” is intended to include “at least one stabilizer” or “one or more stabilizers.”

Any ranges given either in absolute terms or in approximate terms are intended to encompass both, and any definitions used herein are intended to be clarifying and not limiting. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges (including all fractional and whole values) subsumed therein.

Any composition disclosed herein may comprise, consist of, or consist essentially of any element, component and/or ingredient disclosed herein or any combination of two or more of the elements, components or ingredients disclosed herein.

Any method disclosed herein may comprise, consist of, or consist essentially of any method step disclosed herein or any combination of two or more of the method steps disclosed herein.

The transitional phrase “comprising,” which is synonymous with “including,” “containing,” or “characterized by,” is inclusive or open-ended and does not exclude additional, un-recited elements, components, ingredients and/or method steps.

The transitional phrase “consisting of” excludes any element, component, ingredient, and/or method step not specified in the claim.

The transitional phrase “consisting essentially of” limits the scope of a claim to the specified elements, components, ingredients and/or steps, as well as those that do not materially affect the basic and novel characteristic(s) of the claimed invention.

Unless specified otherwise, all molecular weights referred to herein are weight average molecular weights and all viscosities were measured at 25° C. with neat (not diluted) polymers.

As used herein, the term “about” refers to the cited value being within the errors arising from the standard deviation found in their respective testing measurements, and if those errors cannot be determined, then “about” may refer to, for example, within 5% of the cited value.

Furthermore, the invention encompasses any and all possible combinations of some or all of the various embodiments described herein. It should also be understood that various changes and modifications to the presently preferred embodiments described herein will be apparent to those skilled in the art. Such changes and modifications can be made without departing from the spirit and scope of the invention and without diminishing its intended advantages. It is therefore intended that such changes and modifications be covered by the appended claims.

What is claimed is:

1. A method of determining the oxidative stability of a pyrolysis oil, comprising:
heating a sample of the pyrolysis oil to a temperature from about 50° C. to about 220° C.,
passing a stream of air through the sample of pyrolysis oil,
forming a volatile reaction product,
transporting the volatile reaction product into a measurement solution comprising deionized water, and
measuring an electrical conductivity of the measurement solution.

2. The method of claim 1, wherein the pyrolysis oil comprises about 2 wt. % to about 30 wt. % of a C₁-C₄ hydrocarbon gas, about 10 wt. % to about 50 wt. % of a C₅-C₁₅ hydrocarbon oil, about 10 wt. % to about 40 wt. % of a wax, and about 1 wt. % to about 5 wt. % char.

3. The method of claim 1, wherein the pyrolysis oil comprises about 35 wt. % to about 75 wt. % of an olefin and/or a diolefin, about 10 wt. % to about 50 wt. % of a paraffin and/or an iso-paraffin, about 5 wt. % to about 25 wt. % of a naphthene, and about 5 wt. % to about 35 wt. % of an aromatic compound.

4. The method of claim 1, further comprising heating the sample to a temperature from about 90° C. to about 200° C.

5. The method of claim 1, wherein the stream of air is passed at a flow rate from about 1 L/hour to about 20 L/hour.

6. The method of claim 1, wherein the volatile reaction product comprises an organic acid, a peroxide, and any combination thereof.

7. The method of claim 1, wherein the sample excludes biodiesel fuel, a vegetable oil, diesel fuel, gasoline, and/or a lubricating oil.

8. The method of claim 1, wherein the pyrolysis oil further comprises a stabilizer, an antioxidant, a paraffin inhibitor, an asphaltene dispersant, a wax dispersant, a tar dispersant, a neutralizer, a surfactant, a biocide, a preservative, or any combination thereof.

9. The method of claim 1, wherein the electrical conductivity of the measurement solution is measured until an increase in electrical conductivity is detected.

10. A method of determining the ability of an antioxidant to stabilize a pyrolysis oil, comprising:
heating a first sample comprising the pyrolysis oil to a first temperature from about 50° C. to about 220° C.,
passing a first stream of air through the sample of pyrolysis oil,
forming a first volatile reaction product,
transporting the first volatile reaction product into a first measurement solution comprising deionized water,
measuring an electrical conductivity of the first measurement solution for a first period of time until an increase in conductivity is detected,
adding the antioxidant to a second sample of the pyrolysis oil,
heating the second sample to the first temperature,
passing a second stream of air through the second sample,
forming a second volatile reaction product,
transporting the second volatile reaction product into a second measurement solution comprising deionized water,
measuring an electrical conductivity of the second measurement solution for a second period of time until an increase in conductivity is detected, and
comparing the first period of time to the second period of time.

11. The method of claim 10, wherein the pyrolysis oil comprises about 2 wt. % to about 30 wt. % of a C₁-C₄ hydrocarbon gas, about 10 wt. % to about 50 wt. % of a C₅-C₁₅ hydrocarbon oil, about 10 wt. % to about 40 wt. % of a wax, and about 1 wt. % to about 5 wt. % char.

12. The method of claim 10, wherein the pyrolysis oil comprises about 35 wt. % to about 75 wt. % of an olefin and/or a diolefin, about 10 wt. % to about 50 wt. % of a paraffin and/or an iso-paraffin, about 5 wt. % to about 25 wt. % of a naphthene, and about 5 wt. % to about 35 wt. % of an aromatic compound.

13. The method of claim 10, further comprising heating the first sample to a temperature from about 90° C. to about 200° C.

14. The method of claim 10, wherein the first and/or second stream of air is passed at a flow rate from about 1 L/hour to about 20 L/hour.

15. The method of claim 10, wherein the first volatile reaction product and/or the second volatile reaction product comprises an organic acid, a peroxide, and any combination thereof.

16. The method of claim 10, wherein the first sample and the second sample exclude biodiesel fuel, a vegetable oil, diesel fuel, gasoline, and/or a lubricating oil.

17. The method of claim 10, wherein the pyrolysis oil further comprises a stabilizer, an additional antioxidant, a paraffin inhibitor, an asphaltene dispersant, a wax dispersant, a tar dispersant, a neutralizer, a surfactant, a biocide, a preservative, or any combination thereof.

18. The method of claim 10, wherein the antioxidant and/or the additional antioxidant are independently selected from a phenol-based antioxidant and/or an amine-based antioxidant.

19. A method of determining the oxidative stability of a pyrolysis oil, comprising:
introducing a sample of the pyrolysis oil into a sample chamber,
pressurizing the sample chamber to a pressure from about 200 kPa to about 700 kPa with O₂,
heating the sample of the pyrolysis oil to a temperature from about 100° C. to about 180° C.,
monitoring a pressure within the sample chamber, and
determining a time at which a decrease in the pressure within the sample chamber is detected.

20. The method of claim 19, wherein the pyrolysis oil comprises about 2 wt. % to about 30 wt. % of a C₁-C₄ hydrocarbon gas, about 10 wt. % to about 50 wt. % of a C₅-C₁₅ hydrocarbon oil, about 10 wt. % to about 40 wt. % of a wax, and about 1 wt. % to about 5 wt. % char.

21. The method of claim 19, wherein the pyrolysis oil comprises about 35 wt. % to about 75 wt. % of an olefin and/or a diolefin, about 10 wt. % to about 50 wt. % of a paraffin and/or an iso-paraffin, about 5 wt. % to about 25 wt. % of a naphthene, and about 5 wt. % to about 35 wt. % of an aromatic compound.

22. The method of claim 19, wherein the sample excludes biodiesel fuel, a vegetable oil, diesel fuel, gasoline, and/or a lubricating oil.

23. A method of determining the oxidative stability of a pyrolysis oil, comprising:
introducing a sample of the pyrolysis oil into a sample chamber,
pressurizing the sample chamber to a pressure from about 100 psi to about 1,000 psi with O₂,
heating the sample of the pyrolysis oil to a temperature of about 90° C. to about 250° C.,
holding the sample at the pressure and the temperature until an exothermic reaction is detected, and
determining a period of time from a beginning of the pressurizing step to a time when the exothermic reaction is detected.

24. The method of claim 23, wherein the pyrolysis oil comprises about 2 wt. % to about 30 wt. % of a C₁-C₄ hydrocarbon gas, about 10 wt. % to about 50 wt. % of a C₅-C₁₅ hydrocarbon oil, about 10 wt. % to about 40 wt. % of a wax, and about 1 wt. % to about 5 wt. % char.

25. The method of claim 23, wherein the pyrolysis oil comprises about 35 wt. % to about 75 wt. % of an olefin and/or a diolefin, about 10 wt. % to about 50 wt. % of a paraffin and/or an iso-paraffin, about 5 wt. % to about 25 wt. % of a naphthene, and about 5 wt. % to about 35 wt. % of an aromatic compound.

26. The method of claim 23, wherein the sample excludes biodiesel fuel, a vegetable oil, diesel fuel, gasoline, and/or a lubricating oil.
